(19) United States
(12) Patent Application Publication
(10) Pub. No.: US 2025/0332348 A1
(43) Pub. Date: Oct. 30, 2025

(54) An Auto-Injecting Device for Injector

(71) Applicant: Becton, Dickinson and Company, Franklin Lakes, NJ (US)

(72) Inventors: Jian Ma, Suzhou (CN); Wilson Huang, Suzhou (CN); Rongjie Liang, Suzhou (CN); Justin Yang, Shanghai (CN); Jabari Liu, Shanghai (CN)

(21) Appl. No.: 18/870,041

(22) PCT Filed: Jun. 27, 2023
(86) PCT No.: PCT/US2023/026347

§ 371 Date: Nov. 27, 2024

(30) Foreign Application Priority Data
Jun. 29, 2022 (CN) 202210757003.4

(51) Int. Cl. A61M 5/20; A61M 5/46

(52) U.S. Cl. CPC A61M 5/2033; A61M 5/46; A61M 2005/2026

(57) ABSTRACT

The present invention relates to an auto-injecting device for an injector. characterized in that the auto-injecting device comprises an actuating part for actuating the injector and a receiving part for receiving the injector. the actuating part and the receiving part being detachably connected to one other, wherein the actuating part includes: an accumulator being capable of accumulating energy and capable of releasing the accumulated energy: a trigger mechanism for triggering the energy release of the accumulator: and a moving member being capable of moving towards the receiving part when the accumulator releases energy and thus actuating the injector in the receiving part to perform an automatic injection process. The auto-injecting device is capable of, for example, realizing automatic injection of drug in the injector.

### CROSS REFERENCE TO RELATED APPLICATION

This application claims priority to Chinese Patent Application No. 202210757003.4 filed Jun. 29, 2022, the disclosure of which is hereby incorporated by reference in its entirety.

### BACKGROUND OF THE INVENTION

### Field of the Invention

The invention relates to the field of medical instruments, and more particularly relates to an auto-injecting device for an injector.

### Description of Related Art

Injectors are commonly used tools for injecting drugs into human or animal bodies. However, it is difficult for non-professionals other than doctors and nurses to use injectors. On one aspect, it is difficult for a non-professional to manually operate an injector to complete an injection operation; on another aspect, a sharp needle of the injector may injure him accidentally; and besides, a patient may feel afraid of the needle when he makes an injection by himself. Therefore, for example, in the case where self-injection of drug is often needed, such as self-injection of insulin by a diabetic patient, an auxiliary tool that can assist in the completion of drug injection with an injector is very helpful.

An existing auxiliary tool for assisting in drug injection is usually complicated in structure and complicated in operation. Some auxiliary tools can only be used once, so that the cost is high. In addition, an existing auxiliary tool has the possibility of accidental operation, which may lead to the waste of drugs. Also, for prefilled syringes (also called “PFS”), caps thereof are secured too tight to be removed by hand, especially for the elderly. Finally, when using an auxiliary tool, it also makes sense to show the operator the injection status.

### SUMMARY OF THE INVENTION

Therefore, the present invention aims to provide an auto-injecting device for an injector, by means of which at least one of the above-mentioned technical problems existing in the prior art can be solved.

According to one aspect of the present invention, the present invention provides an auto-injecting device for an injector, characterized in that the auto-injecting device comprises an actuating part for actuating the injector and a receiving part for receiving the injector, the actuating part and the receiving part being detachably connected to one other, wherein the actuating part includes an accumulator being capable of accumulating energy and capable of releasing the accumulated energy; a trigger mechanism for triggering the energy release of the accumulator; and a moving member being capable of moving towards the receiving part when the accumulator releases energy and thus actuating the injector in the receiving part to perform an automatic injection process.

Technical effects that can be achieved by the auto-injecting device include, but are not limited to that the auto-injecting device can realize automatic injection of drug in the injector.

Advantageously, the accumulator is capable of accumulating energy by moving the moving member in a direction away from the receiving part.

Advantageously, the actuating part has a support relative to which the moving member is axially movable.

Advantageously, the support is provided with protruding guiding strips, and the moving member is provided with guiding grooves that cooperate with the protruding guiding strips respectively; or, the support is provided with guiding grooves, and the moving member is provided with protruding guiding strips that cooperate with the guiding groove respectively.

Advantageously, the accumulator is configured as a spring, which is arranged between the support and the moving member.

Advantageously, the actuating part includes a housing, and the support is substantially disposed in the housing and is fixedly connected to the housing.

Advantageously, the trigger mechanism is configured as a button having a press portion outside the support and a pivoting portion inside the support, the pivoting portion being pivotably supported on the support.

Advantageously, the button is provided with a retaining portion on the pivoting portion thereof, and the moving member is provided with a tab, the moving member being movable till its tab is positioned above the retaining portion and is retained by the retaining portion.

Advantageously, the actuating part has a press-blocking lever which is switchable between a blocking position preventing the pressing of the button and a releasing position releasing the pressing of the button.

Advantageously, the press-blocking lever is provided with a press-blocking portion, the button having two pivoting portions parallel to each other and spaced apart, and the button is provided with a press-blocking portion between the two pivoting portions, wherein, the press-blocking portion of the press-blocking lever is capable of blocking on the press-blocking portion of the button in the blocking position, and moving to a gap between the two pivoting portions in the releasing position.

Advantageously, a spring is provided between the press-blocking lever and the support, wherein the spring presses the press-blocking lever into its blocking position when no external force is applied to the press-blocking lever, and the press-blocking lever is capable of moving against a spring force of the spring into its releasing position when an external force is applied to the press-blocking lever.

Advantageously, the press-blocking lever comprises an operating portion through which an external force is applicable to the press-blocking lever.

Advantageously, the operating portion is capable of being guided in a guiding groove on the support.

Advantageously, the actuating part comprises a torsion spring pressing against a pressing portion of the button on one hand and the support on the other hand, such that the torsion spring is capable of automatically resetting the button after it is released.

Advantageously, the moving member comprises at least one energy accumulator rod, and the moving member is movable in a direction away from the receiving part by operating the energy accumulator rod.

Advantageously, two opposite energy accumulator rods are provided with a press piece arranged therebetween, and through the press piece the moving member is capable of pressing an injection rod of the injector in a direction toward the receiving part.

Advantageously, in order to detachably connect the actuating part and the receiving part to one other, the actuating part is provided with L-shaped engaging grooves, and the receiving part is provided with locking protrusions configured to cooperate therewith; or, the actuating part is provided with locking protrusions, and the receiving part is provided with L-shaped engaging grooves configured to cooperate therewith.

Advantageously, the receiving part comprises a housing and a receiving sleeve axially movable relative to the housing within the housing, and the receiving sleeve is capable of receiving and supporting an injection tube of the injector.

Advantageously, the receiving sleeve comprises a first flange facing the actuating part, the first flange supporting two horizontal tabs of the injection tube of the injector.

Advantageously, the first flange is provided with a plurality of vertical tabs spaced apart from each other in a circumferential direction, the vertical tabs circumferentially limiting the horizontal tabs of the injection tube.

Advantageously, the receiving sleeve comprises at least two second flanges arranged to be spaced apart successively in an axial direction, and the second flanges are capable of being guided axially in the housing.

Advantageously, the housing is provided with viewing windows, the moving member is provided with marks, and the receiving sleeve is also provided with marks, such that when the injection is completed, the marks on the moving member and the marks on the receiving sleeve are seen simultaneously from the viewing windows.

Advantageously, the receiving part is provided with an injection depth adjustment assembly by means of which a penetration depth of the injection needle of the injector can be preset.

Advantageously, the injection depth adjustment assembly comprises a stopper bulge and a mating stopper that cooperates with the stopper bulge, wherein the mating stopper is provided with a plurality of steps with different heights, and each step is capable of stopping the stopper bulge.

Advantageously, the receiving part comprises a housing and a receiving sleeve axially movable relative to the housing within the housing, and the receiving sleeve is capable of receiving and supporting the injection tube of the injector, the stopper bulge being fixedly disposed on the receiving sleeve, and the mating stopper being disposed circumferentially movable and axially fixed on the housing.

Advantageously, the mating stopper has an operating handle configured to operate from an outer side of the housing, the operating handle being movable within a horizontal slot on the housing so as to align a corresponding step of the mating stopper with the stopper bulge.

Advantageously, a scale is provided aside the horizontal slot on the housing, which scale indicates a presettable penetration depth of the injection needle.

Advantageously, the receiving part comprises a needle shield at a lower end thereof, the needle shield having a needle shielding position protruding from the housing and a retracted position retracted into the housing, wherein the needle shield shields the injection needle of the injector such that it is not seen, and the needle shield is in the retracted position during injection.

Advantageously, the needle shield comprises a barrel portion with both ends open to allow the injection needle to pass therethrough, and in the needle shielding position of the needle shield member, the barrel portion at least circumferentially surrounds a tip of the injection needle of the injector.

Advantageously, the barrel portion comprises a flange capable of stopping on an inner bead of a lower end of the housing within the housing.

Advantageously, the barrel portion is provided with an inner bead at a lower end thereof, and the spring is supported on the inner bead on one hand, and on the receiving sleeve on the other hand, such that the needle shield is always in its needle shielding position when no external force is applied.

Advantageously, the needle shield comprises an elongate trigger piece, the trigger mechanism is configured as a button, and the actuating part comprises a press-blocking lever configured to be switched between an initial blocking position for blocking the pressing of the button and a releasing position for releasing the pressing of the button, wherein the trigger piece extends towards the press-blocking lever and is configured to operate the press-blocking lever to urge it from its blocking position to its releasing position.

Advantageously, when the needle shield is in its needle shielding position, the press-blocking lever is in its blocking position, and when the needle shield is in its retracted position, the press-blocking lever is in its releasing position.

Advantageously, the auto-injection device further comprises a cap removing part for removing a needle cap of the injector, the cap removing part being detachably connected to the receiving part.

Advantageously, the cap removing part comprises a cylindrical connecting portion which is open towards the receiving part and is configured to be fitted around an outer circumferential surface of the receiving part.

Advantageously, the connecting portion is provided with convex points on an inner circumferential surface thereof, and the receiving part is provided with engaging concave portions on an outer peripheral surface thereof; or that, the receiving part is provided with convex points on circumferential surface thereof, and the connecting portion is provided with engaging concave portions on an inner circumferential surface.

Advantageously, the cap removing part comprises an operating flange connected thereto at a lower end of the connecting portion.

Advantageously, the cap removing part has a clamping mechanism for clamping the needle cap of the injector, the clamping mechanism having a plurality of claws arranged one after another in a circumferential direction thereof, and the claws are configured to clamp the needle cap and act on the needle cap from a rear of the needle cap.

Advantageously, the automatic injecting process includes a penetrating process, in which the receiving sleeve moves axially relative to the housing, and a subsequent drug injection process, in which the receiving sleeve does not move axially relative to the housing.

Advantageously, the auto-injection device is reusable.

A person skilled in the art will understand advantages of corresponding embodiments and various additional embodiments by reading the following detailed description of the corresponding embodiments with reference to the drawings as included below.

### BRIEF DESCRIPTION OF THE DRAWINGS

The present invention is further described as below with reference to the accompanying drawings and embodiments, wherein:

FIG. 1A is a schematic side view of an auto-injecting device according to an embodiment of the present invention,

FIG. 1B is another schematic side view of an auto-injecting device as shown in FIG. 1A,

FIG. 1C is a further schematic side view of an auto-injecting device as shown in FIG. 1A,

FIG. 1D is a schematic side view of the auto-injecting device as shown in FIG. 1A, in which the actuating part and the receiving part are separated from one another, and the injection rod of the PFS loaded in the receiving part can be seen,

FIG. 1E is a schematic side view of the auto-injecting device as shown in FIG. 1A. in which the actuating part and the cap removing part are separated from one another.

FIG. 2A is a schematic perspective view of the actuating part of the auto-injecting device as shown in FIG. 1A, in which the energy accumulating rods are not energized,

FIG. 2B is another schematic perspective view of the actuating part of the auto-injecting device as shown in FIG. 2A, in which the energy accumulating rods are energized,

FIG. 2C is a schematic longitudinal sectional view of the actuating part as shown in FIG. 2A.

FIG. 3 is a schematic perspective view of the housing of the actuating part as shown in FIG. 2A,

FIG. 4A is a schematic perspective view of the support of the actuating part as shown in FIG. 2A.

FIG. 4B is another schematic perspective view of the support as shown in FIG. 4A,

FIG. 4C is a schematic side view of the support as shown in FIG. 4A,

FIG. 5A is a schematic perspective view of the moving member of the actuating part as shown in FIG. 2A.

FIG. 5B is a schematic side view of the moving member as shown in FIG. 5A.

FIG. 5C is another schematic side view of the moving member as shown in FIG. 5A,

FIG. 6 is a schematic perspective view of the actuating spring of the actuating part as shown in FIG. 2A,

FIG. 7 is a schematic perspective view of the actuating part as shown in FIG. 2A with its housing hidden,

FIG. 8A is a schematic perspective view of the button of the actuating part as shown in FIG. 2A,

FIG. 8B is a schematic side view of the button as shown in FIG. 8A,

FIG. 9 is a schematic perspective view of the pin of the actuating part as shown in FIG. 2A,

FIG. 10 is a schematic perspective view of the torsional spring of the actuating part as shown in FIG. 2A,

FIG. 11 is a schematic perspective view of the press-blocking lever of the actuating part as shown in FIG. 2A,

FIG. 12 is a schematic partial enlarged view of the button, the moving member and the press-blocking lever of the actuating part as shown in FIG. 2A,

FIG. 13A is a schematic side view of the receiving part of the auto-injecting device as shown in FIG. 1A,

FIG. 13B is a schematic perspective view of the receiving part as shown in FIG. 13A,

FIG. 14A is a schematic perspective view of the housing of the receiving part as shown in FIG. 13A,

FIG. 14B is another schematic perspective view of the housing as shown in FIG. 14A,

FIG. 15A is a schematic perspective view of the receiving sleeve of the receiving part as shown in FIG. 13A,

FIG. 15B is another schematic perspective view of the receiving sleeve as shown in FIG. 15A,

FIG. 16 is a schematic perspective view of the stopper ring of the receiving part as shown in FIG. 13A,

FIG. 17 is a schematic perspective view of the mating stopper of the injection depth adjustment assembly of the receiving part as shown in FIG. 13A, in which the operating handle is not shown,

FIG. 18 is a schematic perspective view of the position of the stop protrusion on the receiving sleeve relative to the mating stopper of the injection depth adjustment assembly,

FIG. 19 is a schematic perspective view of the needle shield of the receiving part as shown in FIG. 13A,

FIG. 20 is a schematic side view of the connected actuating part and receiving part,

FIG. 21 is a schematic longitudinal sectional view of the connected actuating part and receiving part as shown in FIG. 20,

FIG. 22A is a schematic perspective view of the cap removing part of the auto-injecting device as shown in FIG. 1A,

FIG. 22B is a schematic longitudinal sectional view of the cap removing part as shown in FIG. 22A,

FIG. 23 is a schematic perspective view of the PFS about to be fitted into the receiving part and the cap removing part.

FIG. 24A is a schematic longitudinal sectional view of the energy accumulated state of the energy accumulating rods of the actuating part,

FIG. 24B is a schematic longitudinal sectional view of the state of the PFS fitted in the receiving part and the cap removing part,

FIG. 24C is a schematic longitudinal sectional view of the ready-to-connect state of the actuating part and the receiving part,

FIG. 24D is a schematic longitudinal sectional view of the state in which the needle cap of the PFS is removed by using the cap removing part,

FIG. 24E is a schematic longitudinal sectional view of the state in which the auto-injecting device after the cap removing part has been removed therefrom, has just been pressed against an injection region of the human body.

FIG. 24F is a schematic longitudinal sectional view of the state of the auto-injecting device after completion of the penetration process,

FIG. 24G is a schematic longitudinal sectional view of the state of the auto-injecting device after completion of the drug injection process, and

FIG. 24H is a schematic longitudinal sectional view of the state of the auto-injecting device that has left the human body after the injection has been completed.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

Various illustrative embodiments of the present invention are described below. In this description, for the sake of explanation only, various systems, structures and devices are schematically depicted in the drawings, but all the features of actual systems, structures and devices are not described. For example, well-known functions or structures are not described in detail to avoid unnecessary details to obscure the present invention. Of course, it should be understood that in any practical application, many specific implementation decisions need to be made to achieve the specific goals of the developer or user, and the system-related and industry-related restrictions need to be observed. These specific goals may vary with actual applications. In addition, it should be understood that although such specific implementation decisions are complicated and time-consuming, this is a routine task for those of ordinary skill in the art who benefit from the present invention.

The terms and phrases used herein should be understood and interpreted as having a meaning consistent with the understanding of those terms and phrases by those skilled in the relevant art. The consistent usage of terms or phrases herein is not intended to imply a specific definition of the term or phrase, that is, a definition that differs from the ordinary and customary meanings understood by those skilled in the art. For terms or phrases intended to have a special meaning, that is, meanings different from those understood by the skilled person, this special definition will be clearly listed in the description by definition, giving special definition to the term or phrase directly and unambiguously.

Unless the content requires, throughout the following description, the words “comprising” and its variations, such as “including”, will be interpreted in an open and inclusive sense, that is, as “including but not limited to”.

Throughout the description of this description, recitation with reference to such a term as “an embodiment”, “one embodiment”, “some embodiments” “an example” “a specific example” or “some examples”, etc., is intended to mean that a particular feature, structure, material, or characteristic described with reference to the embodiment(s) or example(s) is included in at least one embodiment or example of the present invention. Therefore, phrase “in an embodiment” or “in one embodiment” appearing in various places throughout this description does not necessarily refer to a single embodiment.

Further, specific features, structures, materials or characteristics described may be combined in any one or more embodiments or examples in a suitable manner. Besides, the terms “first”, “second”, and the like are used for descriptive purposes only and cannot be construed as indicating or implying relative importance or indicating implicitly the number of technical features. Thus, features defined as “first”, “second”, etc. may explicitly or implicitly include one or more of the features. In the description of the present invention, the meaning of “a plurality” is two or more, unless otherwise specified.

In the present invention, unless otherwise clearly specified and defined, such terms as “mounted”, “connected”, “coupled”, “connected”, “fixed” and so on should be understood in a broad sense, for example, it can be a fixed connection or it is a detachable connection, or an integral connection; it can be directly connected, or it can be indirectly connected through an intermediate medium, or it can be an internal communication between two components. Those of ordinary skill in the art can understand the specific meanings of the above terms in the present invention according to specific situations.

In the following description of the drawings, like reference numerals refer to like or identical elements throughout the drawings and description thereof. In addition, features in the drawings discussed below are not necessarily drawn to scale. Sizes of the features and elements in the drawings may be appropriately enlarged or reduced to more clearly illustrate the embodiments of the present invention. One may see the relevant prior art for the supplementary aspects of the teachings that can be directly identified from the drawings.

It should be noted here that various modifications and variants in the form and details of the embodiments can be implemented without departing from the general concept of the present invention.

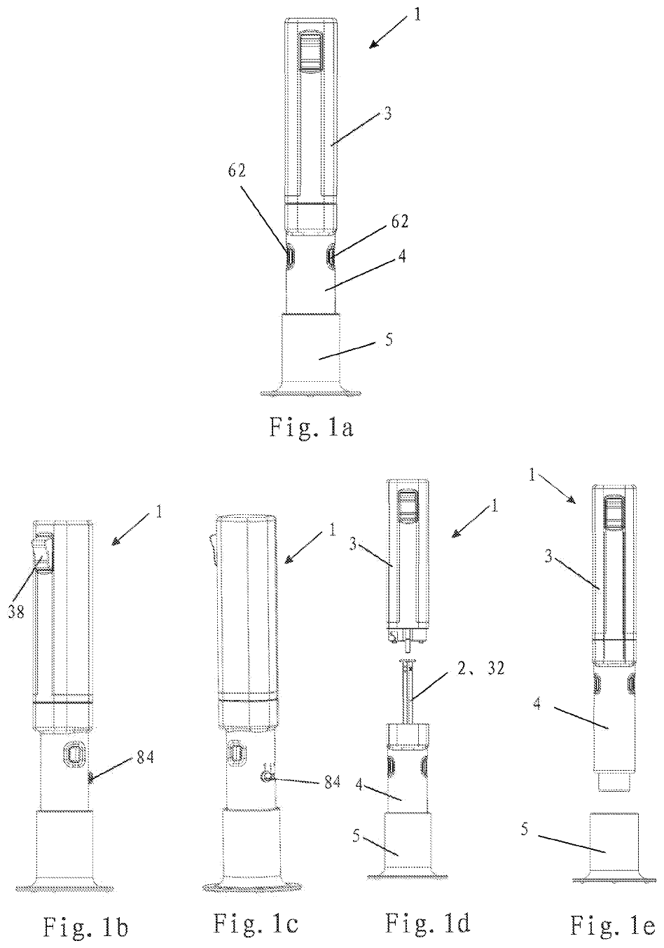

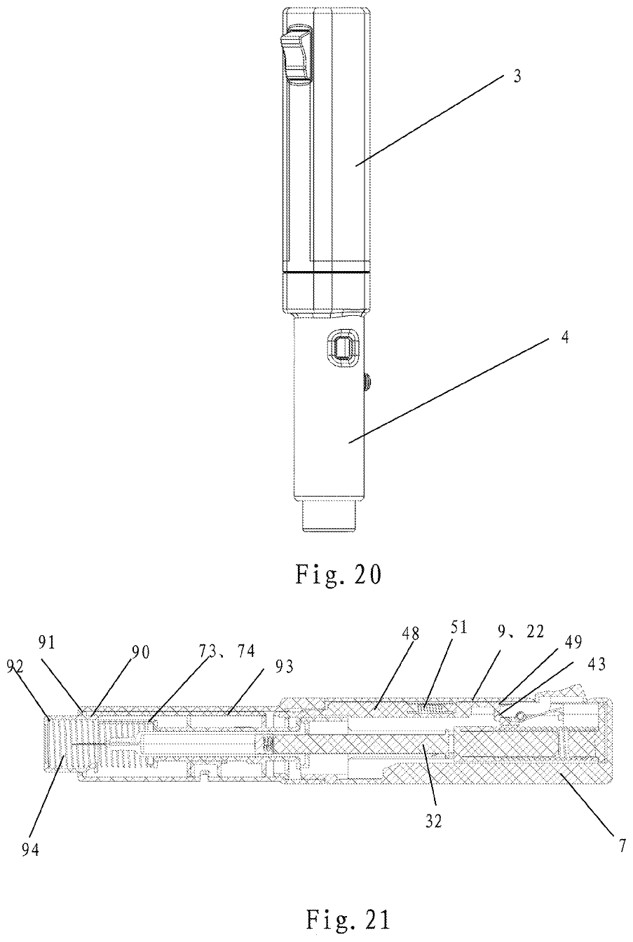

Hereinafter, an exemplary embodiment of an auto-injecting device 1 for an injector 2 according to the present invention will be described with reference to FIGS. 1-24H. The auto-injecting device 1 can be used as an auxiliary tool for assisting in drug injection. Using the auto-injecting device 1, the drug in the injector 2 can be automatically injected into a human body (or an animal body). An operator of the auto-injecting device 1 may be a medical staff in a hospital, or may be a patient himself or any person around him. For example, the patient can use the auto-injecting device 1 to complete the injection by himself anytime and anywhere. The auto-injecting device 1 is reusable and can perform thousands of injections by itself, and only needs to be loaded with a new injector 2 for each injection. The injector 2 may be a fixed dose (e.g. 1 ml or 0.5 ml, etc.) prefilled syringe (hereinafter called “PFS”) 2 or other suitable injector 2 type. Next, the PFS 2 will be described as an example of the injector 2.

Referring to FIGS. 1A-1E, the auto-injecting device 1 may be configured in the form of an injection pen, which can be conveniently operated by the operator's hands. In this embodiment, an auto-injecting device 1 may comprise, from top to bottom, an actuating part 3 for actuating an PFS 2, a receiving part 4 for receiving the PFS 2, and a cap removing part 5 for removing a needle cap 6 of the PFS 2.

In the description herein, orientations “top” and “bottom” related thereto may refer to orientations shown in FIGS. 1A-1E of the auto-injecting device 1 and components thereof. Even though the drawings referred to in the description show their oblique or horizontal orientation, the orientation expressions “top” and “bottom” are still used with reference to orientations shown in FIGS. 1A-1E.

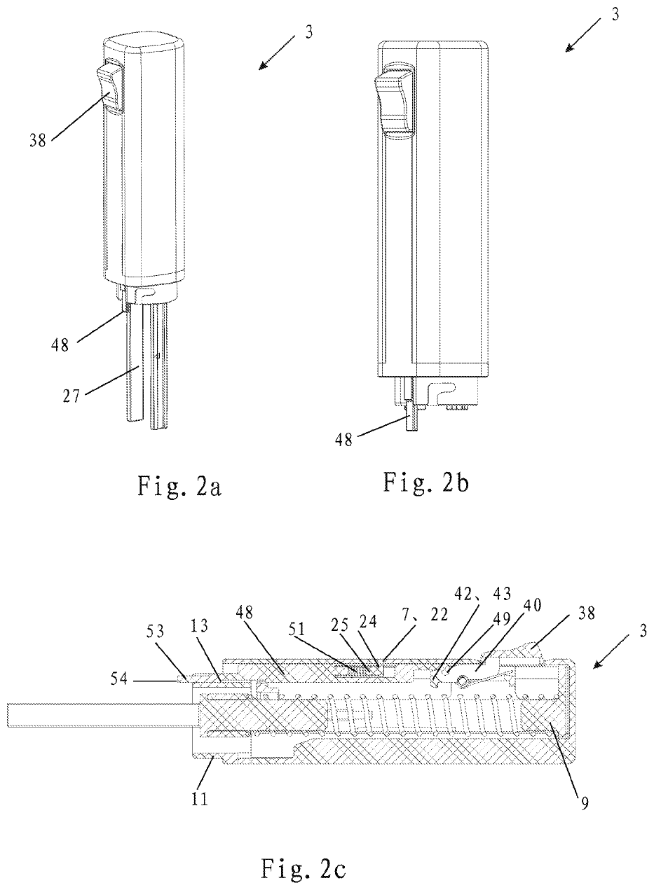

Next, the actuating part 3 of the auto-injecting device 1 and essential components thereof are exemplarily described with reference to FIGS. 2A-12.

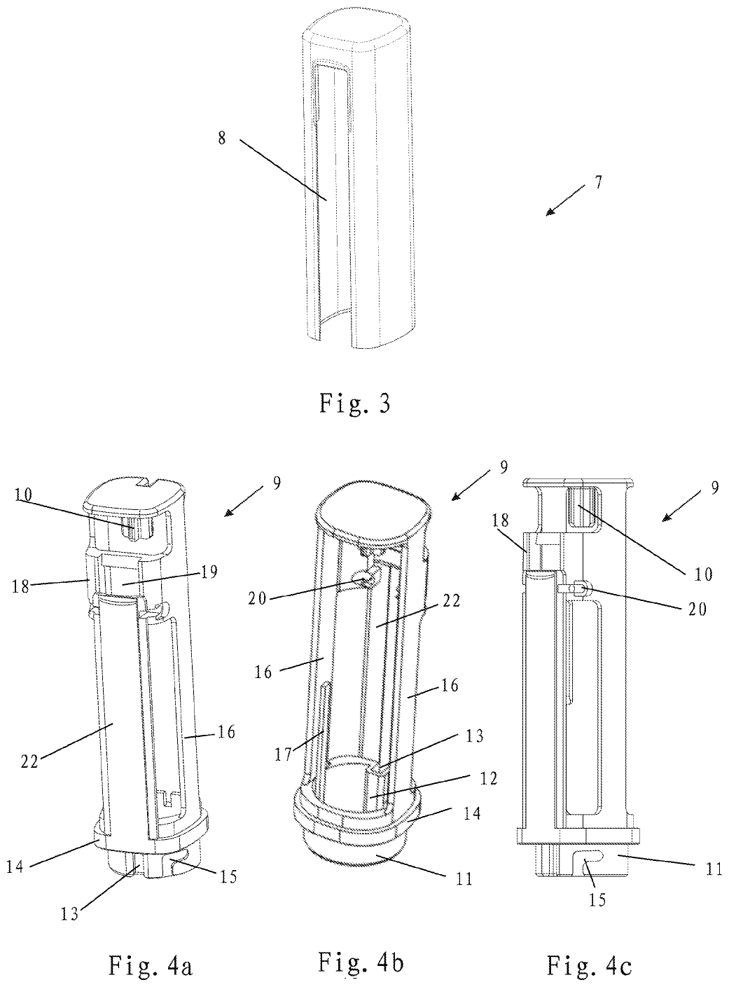

Referring to FIG. 3, the actuating part 3 may comprise a housing 7 which may have a generally clongate cylindrical structure. The housing 7 may be closed at a top end thereof by a top wall and open at a bottom end thereof. The housing 7 may have a rectangular notch 8 on a side wall thereof, which can extend upwards from the bottom end of the housing 7 and end at a distance from the top wall.

Referring to FIGS. 4A-4C, the actuating part 3 may have a support 9 which can be accommodated in the housing 7 and is fixedly connected to the housing 7. The support 9 may have a top wall at an upper end thereof. The top wall may rest with a top face thereof on a bottom face of the top wall of the housing 7. The top wall of the support 9 may be, centrally on a bottom or inner side thereof, provided with, for example integrally formed with a spring strut 10 extending away from the top wall, which spring strut 10 may have a cross-shaped cross-section. Of course, one may anticipate another cross-sectional shape of the spring strut 10, such as a circular or square cross-section.

The support 9 may have, at a lower end thereof, a cylindrical portion 11 that is open at both ends and closed in a circumferential direction. The cylindrical portion 11 may have, in the circumferential direction thereof, a radially inwardly extending bent portion 12 that may form a radially, outwardly open guiding groove 13 having a rectangular cross-section. The cylindrical portion 11 may be provided with a flange 14 on an outer circumferential surface thereof at a substantially middle-up position, and the flange 14 may protrude radially outward from the outer circumferential surface of the cylindrical portion 11. A lower end face of the housing 7 may abut against an upper end face of the flange 14. A lower end of the cylindrical portion 11 may be provided with an L-shaped engaging groove 15 on each of two radially opposite sides, which may respectively include a downwardly open vertical groove portion and a horizontal groove portion connected to an upper end of the vertical groove portion.

The support 9 may have two vertical connecting pieces 16 connecting the top wall at the upper end thereof and the cylindrical portion 11 at the lower end thereof to each other. The vertical connecting pieces 16 can lie opposite one another, here diametrically opposite.

Referring to FIG. 4B, the support 9 may have two protruding guiding strips 17 (only one can be seen here) on an inner side thereof, and each protruding guiding strip 17 may extend parallel to a longitudinal axis of the support 9 on an inner side of one vertical connecting piece 16 and on the inner circumferential surface of the cylindrical portion 11. The support 9 may have, at an upper portion thereof, a horizontal connecting piece 18 connecting the two vertical connecting pieces 16 to each other. The horizontal connecting piece 18 may be provided with a square, circumferentially closed notch 19. The horizontal connecting piece 18 may be provided on an inner side thereof with a pin receptacle 20 having a pin receiving hole on each side of the notch 19. Either of the two pin receptacles 20 of the support 9 may, for example, pivotably accommodate an end of a pin 21 (see FIG. 9).

The support 9 may have a third vertical connecting piece 22, and the third vertical connecting piece 22 may be connected to the horizontal connecting piece 18 and the flange 14 at both ends thereof, respectively. The third vertical connecting piece 22 and the flange 14 can partially close the guiding groove 13 on a radially outer side. A top end of the third vertical connecting piece 22 may extend to a lower side of the notch 19 of the horizontal connecting piece 18. The third vertical connecting piece 22 may be embedded into the notch 8 of the housing 7 in a form-fitting manner, and an outer surface thereof may smoothly transition with an outer circumferential surface of the side wall of the housing 7. Referring to FIG. 2C, the third vertical connecting piece 22 may be provided with a radially inwardly extending tab 24 on an inner side thereof, and below the tab 24, a spring strut 25 extending downward from a lower end surface of the tab 24 is provided.

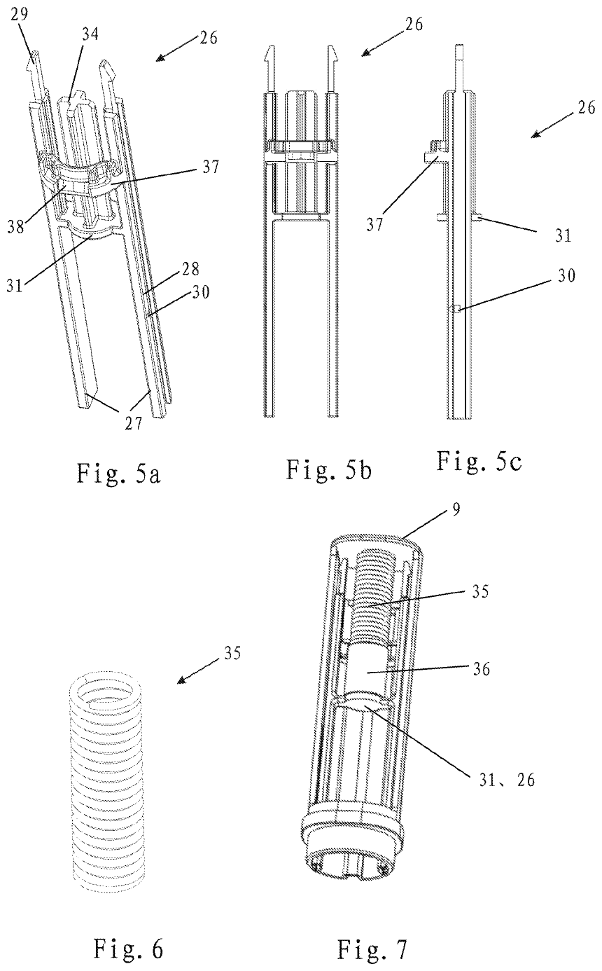

Referring to FIGS. 5A-5C, the actuating part 3 may have a moving member 26 configured to be accommodated in the support 9 and axially movable relative to the support 9. For this purpose, the moving member 26 may have two oppositely, e.g. diametrically, oppositely disposed, energy accumulating rods 27. The energy accumulating rods 27 may be respectively provided with a guiding groove 28 in a longitudinal direction thereof, and the guiding grooves 28 of the two energy accumulating rods 27 can open radially outwardly away from each other. The energy accumulating rods 27 of the moving member 26 may be fitted with their guiding grooves 28 around the protruding guiding strips 17 of the support 9 and be guided by them in an axial direction, such that the moving member 26 is movable axially relative to the support 9 but not rotatable circumferentially. The energy accumulating rods 27 may be respectively provided at a top thereof with a hook 29 formed by a radial projection, which hook 29 is configured to be hooked to a top of one protruding guiding strip 17 to define a maximum downward movement position of the moving member 26 relative to the support member 9. In this maximum downward position of the moving member 26, the energy accumulating rods 27 partially protrude from a lower end of the support 9. By pressing the lower ends of the two energy accumulating rods 27 of the moving member 26 upwards, the moving member 26 can be urged into the support 9 or the housing 7. Referring to FIG. 7, a length of the energy accumulating rods 27 may substantially correspond to a length of the support 9, such that the energy accumulating rods 27 are movable upward relative to the support 9 until the energy accumulating rods 27 are fully received by the support 9. Referring to FIGS. 5A-5C, each energy accumulating rod 27 may be provided with a mark 30 at a lower portion and on an outer side thereof, and the marks 30 may be respectively configured as a structure or a color block that may be easily identified by the operator, the function of which will be described below.

The moving member 26 may have a disc-shaped pressing piece 31, and the disc-shaped pressing piece 31 may be connected at both ends thereof to inner sides facing one another of the two energy accumulator rods 27. The disc-shaped pressing piece 31 may be pressed with a bottom face thereof against an upper end face 33 (see FIG. 23) of the injection rod 32 of the PFS 2 and press the injection rod 32 during injection. The moving member 26 may have a spring strut 34 which may be centrally arranged at a center of the disc-shaped pressing piece 31 and extend upwards, i.e. towards the spring strut 10 on a top wall of the support 9. Likewise, the spring strut 34 may have a cross-shaped cross-section. Of course, one may anticipate another cross-sectional shape of the spring strut 34, such as a circular or square cross-section.

With reference to FIGS. 6 and 7, the actuating part 3 may have an energy accumulator, which can here be designed as an actuating spring 35 configured to be fitted with an upper end thereof around the spring strut 10 of the support 9 and be fitted with a lower portion thereof around the spring strut 34 of the moving member 26. Thereby, when the moving member 26 is pressed upwards relative to the support 9, the actuating spring 35 is further compressed and energized. It can be provided that the actuating spring 35 can still be in a compressed state when the moving member 26 is in the lowermost position relative to the support 9, i.e. when the hooks 29 are resting on the protruding guiding strips 17, such that the actuating spring 35 may always pre-compress the moving part 26 and the support 9 to one another. In order to further compress the actuating spring 35, a sleeve 36 is configured to be fitted around the spring strut 34 of the moving member 26, and a lower portion of the actuating spring 35 is pressed against an upper end face of the sleeve 36. However, the sleeve 36 is not necessary here, and a sufficient force accumulation can be achieved, for example, by selecting a spring having a suitable size and spring rate as the actuating spring 35.

The moving member 26 may have a fan-shaped tab 37, and the fan-shaped tab 37 may be connected at both ends thereof to the two energy accumulator rods 27. The fan-shaped tab 37 may form an inclined surface 38 on an outer side at a central position thereof, and the inclined surface 38 may be oriented upward (specifically, obliquely upward).

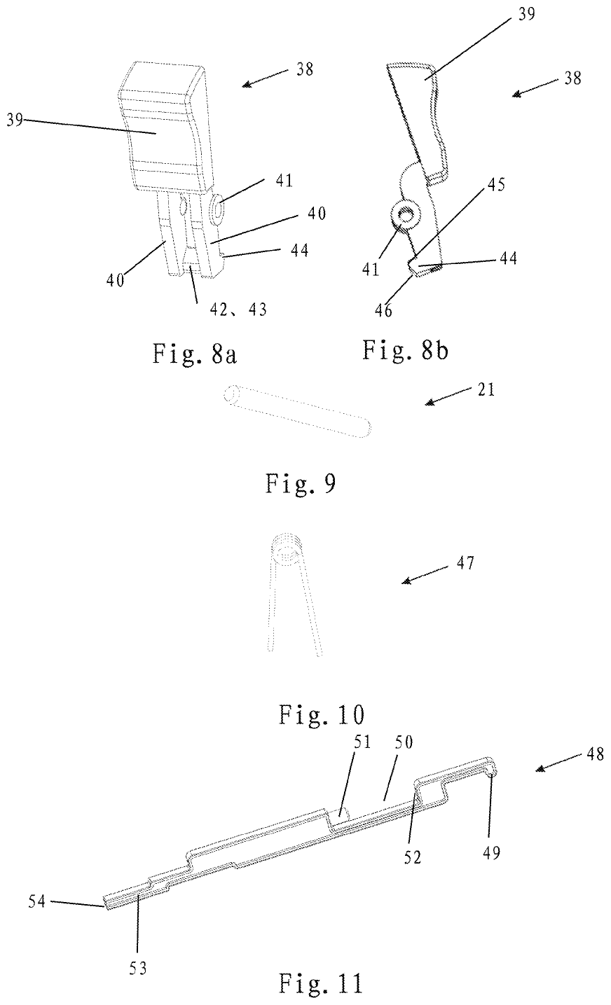

Referring to FIGS. 8A-8B, the actuating part 3 may have a trigger mechanism, which can be configured as a button 38 here, and the button 38 may have a pressing portion 39, which may be received in an upper portion of the notch 8 of the housing 7 and may be pressed from outside. The button 38 may also have two sheet-like pivoting portions 40 that are parallel to each other and spaced a horizontal distance apart, integrally connected to a lower portion of the pressing portion 39. The pivoting portions 40 may protrude into the support 9 from the notch 19 of the support 9, while the pressing portion 39 is still outside the support 9. Each of the pivoting potions 40 may be provided at a substantially central portion thereof with a pin receptacle 41 having a pin receiving hole. The pin 21 can pass through both the pin receiving holes of the pin receptacles 20 of the support 9 and the pin receiving holes of the pin receptacles 41 of the button 38, thereby pivotally connecting the button 38 to the support 9.

Referring to FIG. 8A, the button 38 may have a press-blocking portion 42, which may be disposed at a lower portion thereof between the two pivoting portions 40, while a gap is formed between the two pivoting portions 40 at an upper portion of the press-blocking portion 42. The press-blocking portion 42 may have a press-blocking surface 43 facing radially outward.

Referring to FIG. 8B, the button 38 may have a retaining portion 44 that is radially inward relative to the press-blocking portion 42, the retaining portion 44 may have an upwardly facing retaining surface 45 and a radially inwardly facing, inclined surface 46 that may be oriented downward (specifically, diagonally downward). The inclined surface 46 may cooperate with the inclined surface 38 of the fan-shaped tab 37 of the moving member 26 to facilitate an upward movement of the fan-shaped tab 37 of the moving member 26 over the retaining portion 44 of the button 38.

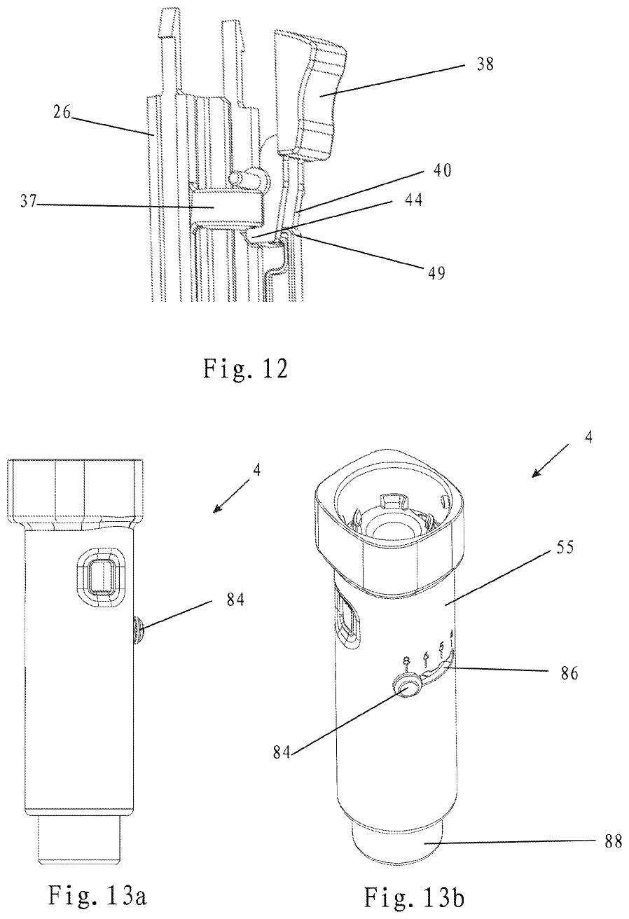

Referring to FIG. 12, as mentioned above, the moving member 26 may move upward relative to the support 9 until the fan-shaped tab 37 of the moving member 26 passes over the retaining portion 44 of the button 38 and is positioned above the retaining portion 44. whereby the lower end face of the fan-shaped tab 37 may abut against the retaining surface 45 of the retaining portion 44 from above, so as to prevent the moving member 26 from being pressed downward by the energized actuating spring 35 when the button 38 is not pressed. If the button 38 is pressed, based on the lever principle, the lower portions of the two pivoting portions 40 and the retaining portion 44 pivot radially outward, such that the retaining surface 45 is radially offset from the lower end surface of the fan-shaped tab 37, thereby allowing the moving member 26 to move downward under a return force of the actuating spring 35.

Referring to FIG. 10, the actuating part 3 may have a torsion spring 47, one end of which may be pressed against an inner side of the pressing portion 39 of the button 38 (see FIG. 2c), and the other end of which may be pressed against the support 9, such that the torsion spring 47 can always press the pressing portion 39 of the button 38 radially outward such that it automatically resets after the pressing is completed.

Referring to FIGS. 2C and 11, the actuating part 3 may have a press-blocking lever 48 provided at an upper end thereof with a press-blocking portion 49 extending radially inward. For example, referring to FIG. 21, in the blocking position of the press-blocking lever 48, the press-blocking portion 49 may be located between the press-blocking surface 43 of the button 38 and an inner circumferential surface of the support 9, thereby preventing the pressing of the button 38. The press-blocking lever 48 is movable upwards from a blocking position thereof to a releasing position as shown in FIG. 2C. In the releasing position of the press-blocking lever 48, the press-blocking portion 49 can be located above the press-blocking surface 43 of the button 38, such that if the button 38 is pressed here, the press-blocking portion 49 may be urged into the gap between the two pivoting portions 40, such that the press-blocking surface 43 of the button 38 may no longer be blocked by the press-blocking portion 49 of the press-blocking lever 48 and may move radially outward to allow the button 38 to be pressed.

The press-blocking lever 48 is provided, below the press-blocking portion 49 thereof, with a recess 50 which is open radially outwardly. The recess 50 is provided, on a lower end surface thereof, with a spring strut 51 extending upward. A preloaded, smaller press-blocking spring 51 (see FIG. 2C) is fitted with two ends thereof around and pre-loaded between the spring strut 51 of the press-blocking lever 48 and the spring strut 25 of the tab 24 of the support 9, and always presses downward, with a return force thereof, the press-blocking lever 48. When no external force is applied to the press-blocking lever 48, the press-blocking lever 48 may always be in the blocking position under action of the return force of the press-blocking spring 51. In the initial blocking position of the press-blocking lever 48, an upper end face 52 of the recess 50 abuts against an upper end face of the tab 24 of the support 9, referring to FIG. 21. In the releasing position of the press-blocking lever 48, the upper end face 52 of the recess 50 moves upward away from the upper end face of the tab 24 of the support 9, referring to FIG. 2C.

The press-blocking lever 48 has an operating portion 53 at a lower end thereof. By operating the operating portion 53, an external force can be applied to the press-blocking lever 48, such that the press-blocking lever 48 is urged from the blocking position into the releasing position against the return force of the press-blocking spring 51. Referring to FIG. 2C, the operating portion 53 of the press-blocking lever 48 can pass through the guiding groove 13 on the cylindrical portion 11 of the actuating part 3 and can be axially guided therein. The actuating portion 53 can extend out of the guiding groove 13 with a lower actuating end 54 thereof.

Next, the receiving part 4 of the auto-injecting device 1 and essential components thereof are exemplarily described with reference to FIGS. 13A-21.

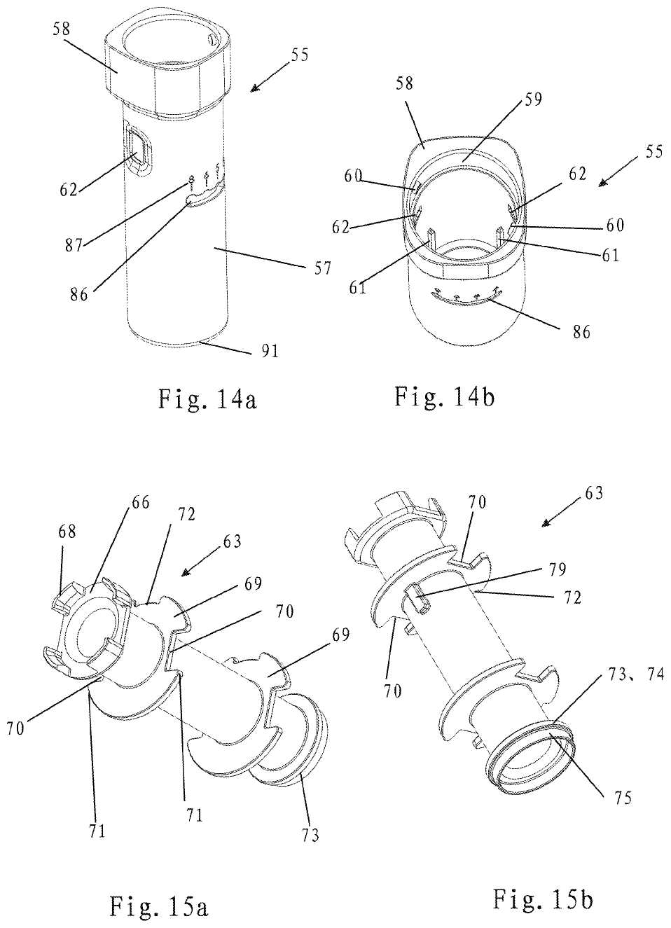

Referring to FIGS. 14A and 14B, the receiving part 4 may comprise a housing 55 which may have a generally clongate cylindrical structure. The housing 55 may have a main body portion 57 and an upwardly open, radially enlarged portion 58 connected to an upper end of the main body portion 57. An upwardly facing transition surface 59 may be formed inside the housing 55 between the main body portion 57 and the radially enlarged portion 58. The housing 55 may be provided with locking protrusions 60 respectively on opposite sides of an inner circumferential surface of the radially enlarged portion 58 thereof, and the locking protrusions 60 may be urged into the horizontal groove portions and be latched there via L-shaped engaging grooves of the cylindrical portion 11 of the actuating part 3, thereby effecting a detachable connection between the receiving part 4 and the actuating part 3. Of course, one may anticipate that the receiving part 4 and the actuating part 3 are detachably connected by another connecting manner, such as by a threaded connection or the like. The housing 55 may be provided with two opposite protruding guiding strips 61 on the inner circumferential surface of the main body portion 57 thereof. The energy accumulating rods 27 of the actuating part 26 may be fitted with the guiding grooves 28 thereof around the protruding guiding strips 61 of the housing 55 respectively and be guided by them in a longitudinal direction, such that the moving member 26 is movable longitudinal relative to the support 55 but not rotatable.

The housing 55 may have two circumferentially spaced viewing windows 62 (sec FIG. 14A) on its main body portion 57 above the two protruding guiding strips 61, through which the operator can view an interior of the housing 55 from outside. When the energy accumulating rods 27 of the moving member 26 move down until their marks 30 reach the viewing windows 62, the marks 30 on the energy accumulating rods 27 can be seen from the viewing windows 62 respectively.

Referring to FIGS. 15A-15B, the receiving part 4 can have a receiving sleeve 63 into which the PFS 2 can be inserted with an injection tube 64 thereof from an upper portion of the receiving sleeve 63. A length of the receiving sleeve 63 may be less than or substantially corresponding to a length of the injection tube 64 of the PFS 2, such that when the PFS 2 is inserted with its injection tube 64 into the receiving sleeve 63, the injection needle 65 of the PFS 2 and a needle cap 6 fitted therearound can protrude from a lower end of the receiving sleeve 63, and an injection rod 32 of the PFS 2 can protrude from an upper end of the receiving sleeve 63.

The receiving sleeve 63 may have an upper flange 66 which may support, with an upwardly facing flange face, two horizontal tabs 67 at a proximal end of the injection tube 64 of the PFS 2 to prevent further entry of the PFS 2 into the receiving sleeve 63. In order to prevent rotation of the injection tube 64 of the PFS 2 relative to the receiving sleeve 63, a plurality of, here four vertical tabs 68 spaced at the same distance, can be arranged one after the other in a circumferential direction on the upper flange 66, and a gap width between adjacent vertical tabs 68 corresponds to a horizontal width of either transverse tab 67 of the injection tube 64, such that the transverse tabs 67 of the injection tube 64 can respectively be latched in two opposite gaps. A diameter of the upper flange 66 may be smaller than an inner diameter of the main body portion 57 of the housing 55 such that the upper flange 66 is movable within the main body portion 57 without interfering therewith.

The receiving sleeve 63 may have two intermediate flanges 69 located below the upper flange 66 and arranged axially spaced one after the other, which two intermediate flanges 69 may have identical geometry. An outer diameter of each intermediate flange 69 may correspond to the inner diameter of the main body portion 57 of the housing 55 such that each intermediate flange 69 may be axially guided to move up and down within the main body portion 57 of the housing 55. Each of the intermediate flanges 69 may respectively have two opposite radially outwardly open notches 70 through which the two energy accumulating rods 27 of the moving member 26 can pass, such that the moving member 26 is movable axially relative to the receiving sleeve 63 without interfering therewith. Marks 71 may be provided on an outer circumferential surface of the upper intermediate flange 69 near the two notches 70, and the marks 71 may be formed as structures or color blocks that can be easily identified by the operator. When the receiving sleeve 63 moves down until its marks 71 reach the viewing windows 62, the marks 71 can be seen from the viewing windows 62. Thus, the viewing windows 62 are positioned and dimensioned to allow both the marks 71 on the upper intermediate flange 69 of the receiving sleeve 63 and the marks 30 on the energy accumulating rods 27 of the moving member 26 to be viewed simultaneously therethrough, e.g. both can be moved to the horizontal and side-by-side alignment. Additionally, each intermediate flange 69 may have a third notch 72.

The receiving sleeve 63 may have a lower flange 73 at a lower end thereof, and a diameter of the lower flange 73 may be smaller than the diameter of either intermediate flange 69. The lower flange 73 may comprise two portions 74, 75 adjacent to each other arranged one after the other in an axial direction, wherein a diameter of the upper flange portion 74 may be larger than a diameter of the lower flange portion 75.

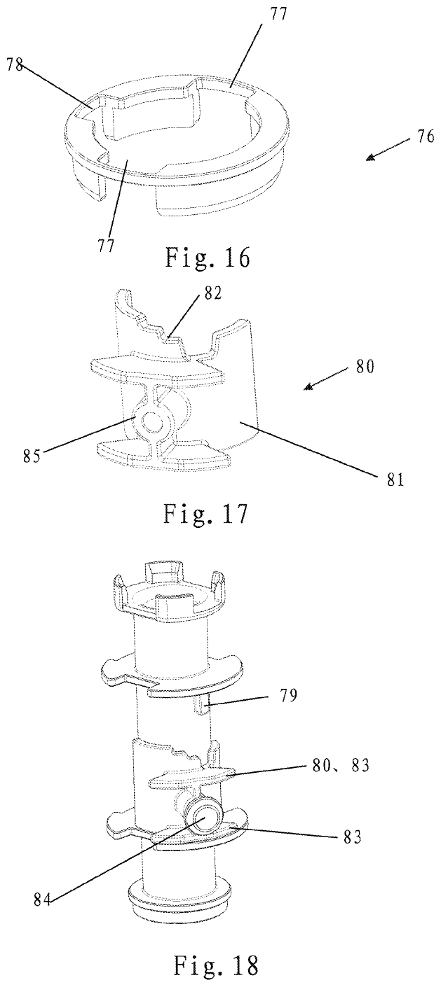

Referring to FIG. 16, the receiving part 4 may have a stop ring 76 which can be supported on the transition surface 59 of the housing 55 and fastened thereto, for example by screws (not shown). The stop ring 76 can stop, with a lower end face thereof, the upper intermediate flange 69 of the receiving sleeve 63, thereby preventing the receiving sleeve 63 from protruding upwards from the housing 55. The stop ring 76 may be provided with two radially, inwardly open recesses 77 through which the energy accumulating rods 27 of the moving member 26 pass, and a radially, inwardly open third recess 78.

Referring to FIGS. 13b, 17 and 18, the receiving part 4 may have an injection depth adjustment assembly, and the injection depth adjustment assembly may include a stopper bulge 79, and the stopper bulge 79 may be provided on the outer circumferential surface of the receiving sleeve 63 underside the upper intermediate flange 69 of the receiving sleeve 63. The injection depth adjustment assembly may further include a mating stopper 80 that cooperates with the stopper bulge 79, and the mating stopper 80 may have an arc-shaped stopper wall 81. and an inner side of the stopper wall 81 may lie against the outer circumferential surface of the receiving sleeve 63. An upper end of the stopper wall 81 may be provided with steps 82 with different heights, for example, with gradually decreasing heights, and four steps 82 with different heights are shown here. Each step 82 can be used to stop the stopper bulge 79 to prevent further downward movement of the receiving sleeve 63, in such a manner as moving the mating stopper 80 relative to the stopper bulge 79 in the circumferential direction. For this purpose, the mating stopper 80 can have two circumferential guide pieces 83 arranged at a distance from one above the other and parallel to each other, each circumferential guide piece 83 can be fixed with a radial inner side thereof on the stopper wall 81 and lie with an arc-shaped radially outer side thereof against the inner wall of the housing 55. Two corresponding cooperating circumferential guide pieces (not shown) may be provided on the inner wall of the housing 55, which may be provided above the upper circumferential guide piece 83 and below the lower circumferential guide piece 83, respectively. The two circumferential guide pieces 83 of the mating stopper 80 can thus be snapped axially in-between the two mating circumferential guide pieces 83 of the housing 55 and can thus be guided in the circumferential direction and locked in the axial direction. The mating stopper 80 can also have a substantially cylindrical operating handle 84, which can be inserted with a radially inner portion thereof into a hole of a projection 85 provided on an outer side of the stopper wall 81, which projection 85 can be arranged between the two circumferential guide pieces 83 of the mating stopper 80. The operating handle 84 can protrude, with a radial outer portion thereof, out of the housing 55, from a horizontal slot 86 provided in the housing 55, for operation by the operator. The operating handle 84 may have a neck portion between its radially inner and radially outer portions, which can be engaged in the horizontal slot 86 and be guided in the circumferential direction therein. Referring to FIG. 13b, a scale 87 may be provided on the outer circumferential surface of the housing 55 above the horizontal slot 86, and an injection depth corresponding to the horizontal position of the operating handle 84 can be read from the scale 87, for example, it is 8 mm, 6 mm, 5 mm, 4 mm here. Referring to FIG. 18, when it is necessary to adjust the penetration depth of the injection needle 65 of the PFS 2 into the human body, it only needs to turn the operating handle 84 horizontally to a desired depth value, whereby the step 82 corresponding to the corresponding height of the mating stopper 80 is moved to just below the stopper bulge 79 of the receiving sleeve 63.

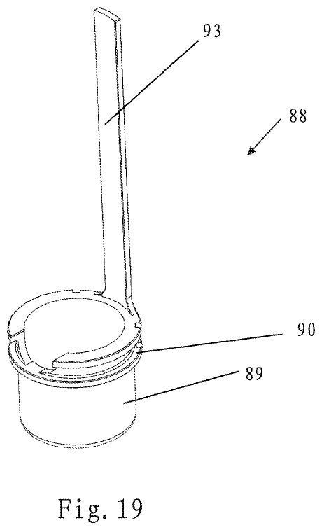

Referring to FIG. 19, the receiving part 4 may have a needle shield 88 provided at a lower end thereof, which can shield the injection needle 65 of the PFS 2 when the injection is not performed, so that the injection needle 65 of the PFS 2 cannot be seen from outside. On the one hand, it can prevent the injection needle 65 from accidentally hurting the human body, and on the other hand, it can prevent the operator from being afraid due to seeing the injection needle 65. The needle shield 88 may have a barrel portion 89 with both ends open, and the barrel portion 89 may have a flange 90 at an upper end thereof, and a lower end of the flange 90 may abut on an inner bead 91 of the lower end of the housing 55 (see FIG. 21) to prevent the barrel portion 89 from fully protruding from the lower end of the housing 55. The barrel portion 89 may have at a lower end thereof an inner bead 92 (see FIG. 21) that defines a diameter larger than the diameter of the needle cap 6 of the PFS 2. The needle shield 88 may also have an clongate trigger piece 93 connected to the flange 90 and extending upward, and the trigger piece 93 may be guided axially within the third notch 78 of each intermediate flange 69 of the receiving sleeve 63 and the third notch 78 of the stop ring 76. Referring to FIG. 21. the trigger piece 93 of the needle shield 88 may be positioned to be disposed just below the press-blocking lever 48, such that when the needle shield 88 is pressed (or urged) upward to move upward, an upper end of the trigger piece 93 can urge the operating end 54 of the operating portion 53 of the press-blocking lever 48 to exert an external force on the press-blocking rod 48, such that the press-blocking rod 48 can move upward against the return force of the press-blocking spring 51 to release the press-blocking to button 38. When the needle shield 88 is not pressed, the upper end of the trigger piece 93 of the needle shield 88 can be spaced apart from the operating end 54 of the operating portion 53 of the press-blocking lever 48 or can be loosely abutted against each other, so that the trigger piece 93 of the needle shield 88 does not exert an external force on the press-blocking lever 48, and the press-blocking lever 48 can be in its blocking position under the action of the return force of the press-blocking spring 51. As described above, in the blocking position of the press-blocking rod 48, the press-blocking rod 48 can block the pressing of the button 38 to prevent the button 38 from being pressed accidentally, so that the auto-injecting device 1 can have a function of preventing the injector 2 from being accidentally triggered or injected.

Referring to FIG. 21, the receiving part 4 may have a needle shield return spring 94, and the needle shield return spring 94 may be supported with a lower end thereof on the inner bead 92 of the lower end of the barrel portion 89 of the needle shield 88, and may be supported with an upper end thereof on the upper flange portion 74 of the lower flange 73 of the receiving sleeve 63. The needle shield return spring 94 can be arranged to always be in a compressed, preloaded state, so that it can always urge the needle shield 88 outward. Only during injection, the needle shield 88 can be pressed upwards into the housing 55 of the receiving part 4 by an injection region of the human body against the return force of the needle shield return spring 94, and then the injection needle 65 of PFS 2 can penetrate into the human body after pressing the button 38. However, once the needle shield 88 leaves the human body, under the action of the return force of the needle shield return spring 94, the needle shield 88 immediately returns to the protruding state from the housing 55 to shield the injection needle 65 therein.

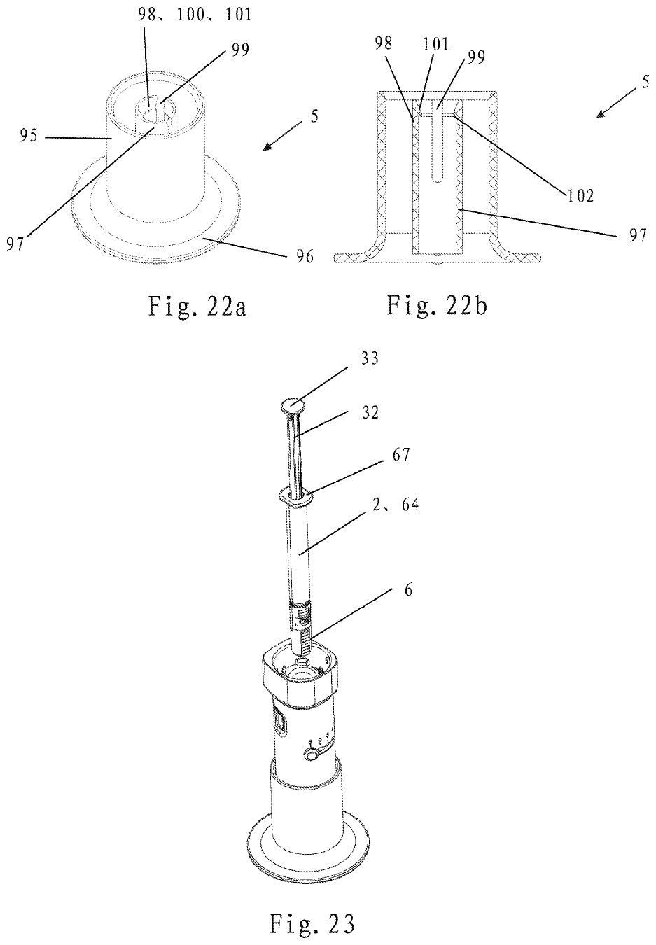

Next, the cap removing part 5 of the auto-injecting device 1 and essential components thereof are exemplarily described with reference to FIGS. 22A-23.

The cap removing part 5 can be used to remove the needle cap 6 of the PFS 2 prior to injection. The cap removing part 5 may have a cylindrical connecting portion 95 which may be open upward and may be fitted over the outer circumferential surface of the lower portion of the housing 55 of the receiving part 4. The connecting portion 95 may be provided with convex points (not shown) on an inner circumferential surface thereof, while engaging concave portions (not shown) on the outer circumferential surface of the lower portion of the housing 55 of the receiving part 4, or vice versa. The convex points and the engaging concave portions may form a male-female fit with each other. When the cap removing part 5 is fitted around the receiving part 4, the convex points can be snapped into the engaging concave portions, and a clicking sound can be generated at the same time to remind the operator that the two have been connected in place.

The cap removing part 5 may be further provided with an operating flange 96 as an operating end connected thereto at a lower end of the connecting portion 95. When it needs to remove the cap removing part 5 from the receiving part 4 so as to remove the needle cap 6 of the PFS 2, the operator can pull the operating flange 96 by hand such that the cap removing operation can be completed easily.

The cap removing part 5 may have a clamping mechanism 97 for clamping the needle cap 6 of the PFS 2, and the clamping mechanism 97 may be coaxially arranged with the connecting portion 95 and, for example, may substantially axially overlap with the connecting portion 95. The clamping mechanism 97 may have a plurality of, here, three claws 98 arranged one after another at an upper portion in a circumferential direction thereof. Adjacent jaws 98 can form therebetween a slot 99 that opens upward, thereby allowing each jaw 98 to expand radially outward when subjected to a radial force. An upper end of each jaw 98 may be provided with a boss 100 extending radially inward beyond an inner side of the jaw 98, and an inner surface of the boss is formed as an upward inclined plane 101 and a downward horizontal bottom surface 102. When the needle cap 6 of the PFS 2 is inserted into the cap removing part 5, the inclined surface 101 of the boss 100 can facilitate the needle cap 6 to urge each jaw 98 radially outward and into the clamping mechanism 97. After the needle cap 6 completely enters an interior of the clamping mechanism 97, the claws 98 can move radially inward based on an clastic return force and abut with a horizontal bottom surface 102 against the upper end face of the needle cap 6, such that when the cap removing part 5 is removed, the claws 98 can remove the needle cap 6 from the PFS 2 with their bosses 100. An inner diameter defined by an inner side of the claws 98 may be slightly smaller than the diameter of the needle cap 6 of the PFS 2, so as to hold the needle cap 6 in it more tightly.

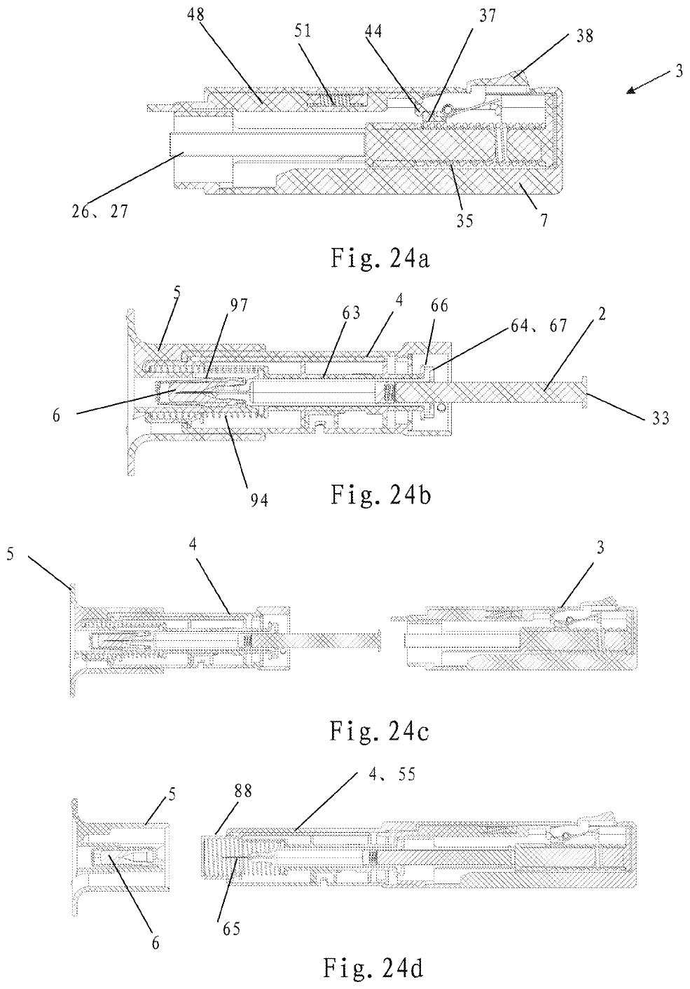

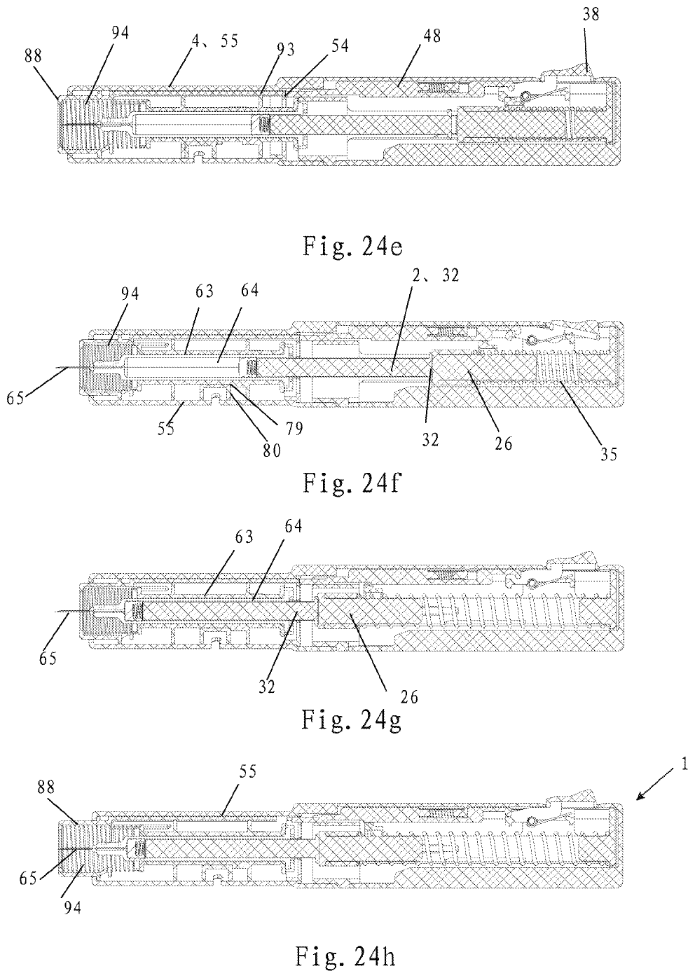

Next, the operation process and working principle of the auto-injecting device 1 according to this embodiment will be illustrated with reference to FIGS. 24A-24H.

Step 1: preparing an auto-injecting device 1 and a PFS 2.

Step 2: separating an actuating part 3 and a receiving part 4 of the auto-injecting device 1 from one another.

Step 3: referring to FIG. 24A, pressing energy accumulating rods 27 of a moving member 26 of the actuating part 3 into a housing 7 of the actuating part 3, such that an actuating spring 35 is compressed and energized. Whereby, a lower end face of a fan-shaped tab 37 of the moving member 26 abuts against a retaining surface 45 of a retaining portion 44 of a button 38 from above, such that the moving member 26 is retained by the button 38, and a press-blocking lever 48 is in a blocking position thereof based on a return force of a press-blocking spring 51, so as to prevent accidental release of the moving member 26 by accidentally pressing the button 38.

Step 4: refer to FIG. 24B, fitting the PFS 2 into the receiving part 4. Whereby, two horizontal tabs 67 of an injection tube 64 of the PFS 2 abut against an upper flange 66 of a receiving sleeve 63, a needle cap 6 of the PFS 2 is inserted into a clamping mechanism 97 of a cap removing part 5, and a needle shield return spring 94 is in an initial state thereof with a maximum length.

Step 5: referring to FIG. 24C, connecting the actuating part 3 and the receiving part 4 to one another.

Step 6: operating an operating handle 84 of an injection depth adjusting assembly to adjust a desired injection depth.

Step 7: referring to FIG. 24D, removing the cap removing part 5 from the receiving part 4 so as to remove the needle cap 6 of the PFS 2. Whereby, a needle shield 88 of the receiving part 4 still protrudes from the housing 55, such that the injection needle 65 cannot be seen from outside.

Step 8: referring to FIG. 24E, pressing the auto-injecting device 1 after the cap removing part has been removed therefrom, with a lower end thereof, against an injection region of a human body. Whereby, the needle shield 88 can be urged against an elastic force of the needle shield return spring 94 into the housing 55 as being pressed into the housing 55 of the receiving part 4 by the injection region of the human body, and at the same time, a trigger piece 93 on the needle shield 88 presses with an upper end thereof and moves an lower operating end 54 of a press-blocking lever 48 upward, such that the press-blocking lever 48 moves upward from a blocking position thereof to a releasing position thereof, such that the button 38 is in a pressable state thereof.

Step 9: pressing the button 38 to release the moving member 26 to start an automatic injection process.

The automatic injection process can be divided into two stages, namely, a penetrating process and a drug injection process.

After the button 38 is pressed, the penetrating process is automatically performed first. Referring to FIG. 24F, the moving member 26 moves downward under a return force of the actuating spring 35. The moving member 26 presses an upper end face 32 of an injection rod 32 of the PFS 2 and drives the entire PFS 2 to move downwards. An injection needle 65 starts to penetrate into the human body, and an injection tube 64 brings the receiving sleeve 63 to move downwards together against an elastic force of the needle shield return spring 94. The penetrating process is performed until a stopper bulge 79 of a depth adjustment assembly provided on the receiving sleeve 63 stops on a corresponding step 82 of a mating stopper 80 provided on the housing 55 of the receiving part 4, thereby reaching a predetermined penetrating depth, such as the completion state of the penetrating process as shown in FIG. 24F. When the penetrating process is completed, marks 71 on the receiving sleeve 63 have moved into viewing windows 62 and can be seen by the operator. During the penetrating process, the injection rod 32, the injection tube 64, the injection needle 65, and the moving member 26 and the receiving sleeve 63 of the PFS 2 move downward together, while the injection rod 32 and the moving member 26 do not or almost do not move relative to the injection tube 64 and the receiving sleeve 63 respectively. The needle shield return spring 94 can also play a role in buffering the penetrating impact during the penetrating process.

The drug injection process is automatically performed immediately after the penetrating process. Referring to FIG. 24G, during the drug injection process, the receiving sleeve 63 and therefore the injection tube 64 and the injection needle 65 can no longer move downward due to the stop of the depth adjustment assembly, and the moving member 26 can continue to press the injection rod 32 to move downward, such that the injection rod 32 injects drug into the human body. The drug injection process is performed until a lower end face of the injection rod 32 stops against a lower end face of an inner cavity of the injection tube 64, such that the injection rod 32 completely urges the drug from the injection tube 64 into the human body, see the state where the drug injection process is completed as shown in FIG. 24g. When the drug injection process is completed, marks 30 on energy accumulating rods 27 of the moving member 26 also move into the viewing windows 62 and can be seen by the operator, whereby the marks 71 on the receiving sleeve 63 and the marks 30 on the energy accumulating rods 27 of the moving member 26 can be positioned horizontally, side by side or aligned with each other, thereby indicating to the operator that the drug injection process has been completed.

Step 10: referring to FIG. 24H, pulling out the injection needle 65 from the human body. Whereby, the needle shield 88 is automatically and quickly ejected from the housing 55 based on the return force of the needle shield return spring 94 so as to shield the injection needle 65. At this point, the injection is completed. During the whole injection process, the operator did not see the injection needle 65.

Step 11: separating the actuating part 3 and the receiving part 4 of the auto-injecting device 1 from one another again, and emptying the used PFS 2 out of the receiving part 4 as garbage, and then connecting the actuating part 3, the receiving part 4 and the cap removing part 5 of the auto-injecting device 1 to one another for the next injection.

A single use process of the auto-injecting device 1 has been described above, but the auto-injecting device 1 according to the present invention can repeat the above-mentioned use process many times to realize the feature of reusability.

Advantages of the auto-injecting device 1 according to the present invention include, for example: the auto-injecting device 1 can realize automatic injection of the drug in an injector, and can be reused; the auto-injecting device 1 has a simple structure and a smaller number of components, and of some of these components, a single component can carry out different functions. The auto-injecting device 1 is provided with a press-blocking mechanism 48. 93 to prevent an operator from accidental operation on the button 38; the auto-injecting device 1 is provided with a needle shield mechanism 89, 94, which can prevent the injection needle 65 from accidentally hurting the human body and helps the operator overcome the fear of needles; the auto-injecting device 1 is provided with a cap removing mechanism 5 for the needle cap 6 of the PFS 2, which can easily remove the needle cap 6 of the PFS 2 that is difficult to be removed with hand only; the auto-injecting device 1 is provided with an injection process viewing mechanism 62, 30, 71 that can show the operator that the injection is completed.

The present invention may include any feature or combination of features implicitly or explicitly disclosed herein or a generic concept thereof, and is not limited to any defined scope as listed above. Any elements, features and/or structural arrangements described herein may be combined in any suitable manner.

The specific embodiments disclosed above are merely exemplary, and it will be apparent to those skilled in the art who benefit from the teachings herein that the present invention can be modified and implemented in different but equivalent manners. It is therefore obvious that changes and modifications can be made to the specific embodiments as disclosed above, and all these variations are considered to fall within the scope and spirit of the present invention.

1. An auto-injecting device for an injector, characterized in that the auto-injecting device comprises an actuating part for actuating the injector and a receiving part for receiving the injector, the actuating part and the receiving part being detachably connected to one other, wherein the actuating part includes:
an accumulator being capable of accumulating energy and capable of releasing the accumulated energy;
a trigger mechanism for triggering the energy release of the accumulator; and
a moving member being capable of moving towards the receiving part when the accumulator releases energy and thus actuating the injector in the receiving part to perform an automatic injection process wherein the trigger mechanism is configured as a button having a press portion outside the support and a pivoting portion inside the support, the pivoting portion being pivotably supported on the support,
wherein the actuating part has a press-blocking lever which is switchable between a blocking position preventing the pressing of the button and a releasing position releasing the pressing of the button, and
wherein the press-blocking lever is provided with a press-blocking portion, the button having two pivoting portions parallel to each other and spaced apart.

2. The auto-injecting device according to claim 1, characterized in that the accumulator is capable of accumulating energy by moving the moving member in a direction away from the receiving part.

3. (canceled)

4. The auto-injecting device according to claim 1, characterized in that the support is provided with protruding guiding strips, and the moving member is provided with guiding grooves that cooperate with the protruding guiding strips respectively; or, the support is provided with guiding grooves, and the moving member is provided with protruding guiding strips that cooperate with the guiding groove respectively.

5. The auto-injecting device according to claim 1, characterized in that the accumulator is configured as a spring, which is arranged between the support and the moving member.

6. The auto-injecting device according to claim 1, characterized in that the actuating part includes a housing, and the support is substantially disposed in the housing and is fixedly connected to the housing.

7. (canceled)

8. The auto-injecting device according to claim 1, characterized in that the button is provided with a retaining portion on the pivoting portion thereof, and the moving member is provided with a tab, the moving member being movable till its tab is positioned above the retaining portion and is retained by the retaining portion.

9. (canceled)

10. (canceled)

11. The auto-injecting device according to claim 1, characterized in that a spring is provided between the press-blocking lever and the support, wherein the spring presses the press-blocking lever into its blocking position when no external force is applied to the press-blocking lever, and the press-blocking lever is capable of moving against a spring force of the spring into its releasing position when an external force is applied to the press-blocking lever.

12. The auto-injecting device according to claim 11, characterized in that the press-blocking lever comprises an operating portion through which an external force is applicable to the press-blocking lever.

13. The auto-injecting device according to claim 11, characterized in that the operating portion is capable of being guided in a guiding groove on the support.

14. The auto-injecting device according to claim 1, characterized in that the actuating part comprises a torsion spring pressing against a pressing portion of the button on one hand and the support on the other hand, such that the torsion spring is capable of automatically resetting the button after it is released.

15. The auto-injecting device according to claim 2, characterized in that the moving member comprises at least one energy accumulator rod, and the moving member is movable in a direction away from the receiving part by operating the energy accumulator rod.

16. The auto-injecting device according to claim 15, characterized in that two opposite energy accumulator rods are provided with a press piece arranged therebetween, and through the press piece the moving member is capable of pressing an injection rod of the injector in a direction toward the receiving part.

17. The auto-injecting device according to claim 1, characterized in that in order to detachably connect the actuating part and the receiving part to one other, the actuating part is provided with L-shaped engaging grooves, and the receiving part is provided with locking protrusions configured to cooperate therewith; or, the actuating part is provided with locking protrusions, and the receiving part is provided with L-shaped engaging grooves configured to cooperate therewith.

18. The auto-injecting device according to claim 1, characterized in that the receiving part comprises a housing and a receiving sleeve axially movable relative to the housing within the housing, and the receiving sleeve is capable of receiving and supporting an injection tube of the injector.

19. The auto-injecting device according to claim 18, characterized in that the receiving sleeve comprises a first flange facing the actuating part, the first flange supporting two horizontal tabs of the injection tube of the injector.

20. The auto-injecting device according to claim 19, characterized in that the first flange is provided with a plurality of vertical tabs spaced apart from each other in a circumferential direction, the vertical tabs circumferentially limiting the horizontal tabs of the injection tube.

21. The auto-injecting device according to claim 18, characterized in that the receiving sleeve comprises at least two second flanges arranged to be spaced apart successively in an axial direction, and the second flanges are capable of being guided axially in the housing.

22. The auto-injecting device according to claim 18, characterized in that the housing is provided with viewing windows, the moving member is provided with marks, and the receiving sleeve is also provided with marks, such that when the injection is completed, the marks on the moving member and the marks on the receiving sleeve are seen simultaneously from the viewing windows.

23. The auto-injecting device according to claim 1, characterized in that the receiving part is provided with an injection depth adjustment assembly by means of which a penetration depth of the injection needle of the injector can be preset.

24. The auto-injecting device according to claim 23, characterized in that the injection depth adjustment assembly comprises a stopper bulge and a mating stopper that cooperates with the stopper bulge, wherein the mating stopper is provided with a plurality of steps with different heights, and each step is capable of stopping the stopper bulge.

25.-40. (canceled)
